(19) United States
(12) Patent Application Publication
(10) Pub. No.: US 2004/0022671 A1
(43) Pub. Date: Feb. 5, 2004

(54) Object decontaminator

(76) Inventor: James Malatesta, Hockessin, DE (US)

Correspondence Address: SCHNADER HARRISON SEGAL & LEWIS, LLP, 1600 MARKET STREET, SUITE 3600, PHILADELPHIA, PA, 19103

(21) Appl. No.: 10/310,317

(22) Filed: Dec. 5, 2002

Related U.S. Application Data
(60) Provisional application No. 60/336,822, filed on Dec. 5, 2001.

(51) Int. Cl. A01N 1/00; A61L 2/00

(52) U.S. Cl. 422/28; 422/32; 422/33; 422/292; 422/298

(57) ABSTRACT

A decontaminator and methods of decontamination of objects within a chamber, or the chamber itself. The decontaminator includes a decontaminant container for storing decontaminant. The container is connected to a valve to regulate the flow of decontaminant out of the container and into a vapor box or directly into a chamber. A heater heats the decontaminant in the vapor box to form a vapor. A fan may be implemented to disburse the vapor into the chamber. A decontaminant distribution device may also be included to improve circulation of the decontaminant within the chamber. A decontamination method is also disclosed.

This application is based on provisional application having serial No. 60/336,822 having a filing date of Dec. 5, 2001 entitled MAILBOX DECONTAMINATOR.

### FIELD OF THE INVENTION

The invention relates to bio-deactivation, and more particularly to decontamination of enclosures, and objects contained therein.

### BACKGROUND OF THE INVENTION

The presence of biological contaminants in the United States mail system has created a need for decontamination of the mail. Currently, decontamination is only performed at select mail sites, and only with respect to a portion of the mail. Therefore, it is desirable for mail recipients to have the capability of decontaminating their mail.

Traditional decontamination methods are labor-intensive, and often require significant down times of equipment and facilities.

Decontamination can be accomplished by spraying and wiping potentially contaminated surfaces. When dealing with bio-contamination, however, it is exceedingly difficult to remove all contaminants from every crevice.

Chlorine dioxide bomb or peracetic acid may also be used to decontaminate surfaces. More widely used is paraformaldehyde which is typically evaporated in electric skillets and disbursed into hard to reach areas with fans. Although effective, extensive time and effort is required to neutralize the gases before they may be vented to the outdoors. Use of such hazardous materials pose risks in and of themselves.

Hydrogen peroxide vapor has been used to decontaminate chambers, such as isolators and freeze dryers but is not currently used by mail recipients to decontaminate their mail.

The incidence of bio-contaminated mail has created a need to modify and apply hydrogen peroxide and other chemical decontamination methods for use by mail recipients. Furthermore, there is a need for systems that may be used to decontaminate other types of chambers and objects therein.

### SUMMARY OF THE INVENTION

Embodiments of the invention include a decontaminator and methods of decontamination of objects within a chamber, or the chamber itself. The decontaminator includes a decontaminant container for storing decontaminant. The container is connected to a valve to regulate the flow of decontaminant out of the container and into a vapor box or directly into a chamber. A heater can be used to heat the decontaminant in the vapor box to form a vapor. Optionally, a fan may be implemented to disburse the vapor into the chamber. A decontaminant distribution device may also be included to improve circulation of the decontaminant within the chamber.

### DESCRIPTION OF THE DRAWING

The invention is best understood from the following detailed description when read with the accompanying drawings.

The invention is best understood from the following description when read with the accompanying drawing.

The FIGURE depicts an object decontaminator according to an illustrative embodiment of the invention.

### DESCRIPTION OF THE INVENTION

Embodiments of the present invention provide a mailbox decontaminator that can be used at Postal Service delivery points, or other mail carrier deliver points. Although the invention is particularly suitable for use at delivery points, it can be used at any point in a mail delivery process. The methods and systems of the present invention may also be applied to decontamination of documents or other objects that do not necessarily relate to the postal system. Advantageously the invention may be implemented economically and on a small scale basis.

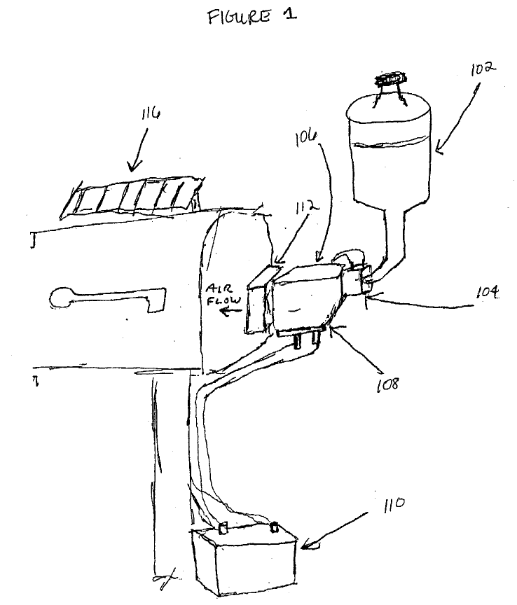

FIG. 1 depicts a decontamination system according to an illustrative embodiment of the invention. Hydrogen peroxide or other decontaminant is placed in a holding tank 102. Holding tank 102 may be any container suitable for storing the decontaminant. The decontaminant holding tank 102 is preferably outfitted with a locking device to maintain control of substances contained therein. Flow of the decontaminant from holding tank 102 may be controlled by a valve 104 such as a solenoid valve. Other electronic or manual valves and sensors may be incorporated to control the flow of the decontaminant.

In an exemplary embodiment of the invention, the decontaminant travels via gravity to a vapor box 106. The scope of the invention, however, includes the decontaminant being forced into vapor box 106 by means other than gravity. The decontaminant enters vapor box 106, preferably until it is one-third full. Valve 104 can be activated and deactivated by a timing control relay. The apparatus may be controlled by a power supply 110, which may be for example, a 12-volt DC battery. In an illustrative embodiment of the invention, a solar collector 116 is used to keep power supply 110 charged.

The decontaminant can be vaporized by a heater 108 supplying heat to vapor box 106. Preferably the heater is controlled by a thermostat to provide the most effective temperature. Optionally, a fan 112 propels the vapor into a chamber 114, such as a mailbox. Other devices for propelling vapor into chamber 114 are within the spirit and scope of the invention. A further example of such a device is a ring compressor or other type of compressor.

Chamber 114 is preferably closed with an electromagnetic latch, to keep chamber 114 shut until the decontamination process is completed. In an illustrative embodiment of the invention, fan 112 is a 12-volt DC thermostatic fan. Although it is preferable to have a fan 112, the invention may operate in certain circumstances without one. Additional vapor propelling devices and vapor boxes can be incorporated which may be beneficial for large chambers. Vapor propelling devices may be switched off by a timing control relay.

The decontaminant vapor settles over objects such as mail in chamber 114, thereby decontaminating the items. To further distribute the decontaminant, the objects may be vibrated or otherwise moved within the chamber. This may be achieved by a decontaminant distribution device that moves objects directly, imparts movement to the objects by forces applied to chamber 114, or a combination thereof. The decontaminant distribution device may be mechanical or electrical. In an exemplary embodiment of the invention, the decontaminant distribution device vibrates chamber 114. This may be accomplished, for example, with a motor in functional cooperation with a pulley applied off-center of chamber 114.

It is noted that an object to be decontaminated may be the chamber itself. Accordingly, the methods and apparatus described herein can be used to create a substantially contaminant-free environment in which objects can be placed.

Any decontaminant that can be used according to the methods and apparatuses described herein, is within the spirit and scope of the invention. In an exemplary embodiment of the invention, the decontaminant is one that destroys anthrax. Illustrative examples of decontaminants include peracetic acid, peroxides, such as hydrogen peroxide, chlorine dioxide, and formaldehydes, such as hydroformaldehyde and paraformaldehyde.

It is noted that the decontaminant maybe a gas at atmospheric pressure. In such cases, there is no need to heat the decontaminant. Furthermore, the decontaminant may flow to chamber 114 without first entering a vapor box.

Although the illustrative embodiment of FIG. 1 shows an automated or electronic system, some or all of the operation steps may be performed manually. For example, in place of a solenoid, a manually operated valve may be incorporated.

Activation of the system may be accomplished via a switch, such as a toggle switch, or upon closure of a chamber door. Additionally, sensors may be incorporated to assure that the system is only activated when desired, such as when objects are in the chamber.

Indicator lights, or other signals may be incorporated into the system to alert a user to processes in progress or completed, or maintenance required, such as refilling the decontaminant holding tank.

The decontamination system may be housed in an enclosure to protect system components and provide system security.

The invention further includes a method of decontaminating a chamber, and/or objects in a chamber, using a system as described herein.

While the invention has been described by illustrative embodiments, additional advantages and modifications will occur to those skilled in the art. Therefore, the invention in its broader aspects is not limited to specific details shown and described herein. Modifications, for example, to types of decontaminants, component configurations, individual components, and objects to be decontaminated, may be made without departing from the spirit and scope of the invention. Accordingly, it is intended that the invention not be limited to the specific illustrative embodiments, but be interpreted within the full spirit and scope of the appended claims and their equivalents.

Claimed is:

1. A decontaminator comprising:
a decontaminant container;
a valve connected to the decontaminant container to regulate flow of decontaminant from the decontaminant container;
a vapor box connected to the valve; and
a heater to heat the decontaminant in the vapor box to form a vapor.

2. The decontaminator of claim 1 further comprising a vapor propelling device to disburse the vapor into a chamber.

3. The decontaminator of claim 1 further comprising a solar collector to power at least a portion of the decontaminator.

4. The decontaminator of claim 1 wherein the valve is controlled by a timing control relay.

5. The decontaminator of claim 2 wherein the vapor propelling device is a thermostatic fan.

6. The decontaminator of claim 2 further comprising a timing relay functionally connected to the fan.

7. The decontaminator of claim 1 further comprising a chamber into which decontaminant is dispersed.

8. The decontaminator of claim 7 wherein the chamber is closed with an electromagnetic latch.

9. The decontaminator of claim 1 further comprising a decontaminant.

10. The decontaminator of claim 9 wherein the decontaminant is selected
from the group consisting of peracetic acid, peroxides, chlorine dioxide, and formaldehydes.

11. The decontaminator of claim 9 wherein the decontaminant is hydrogen peroxide.

12. The decontaminator of claim 9 wherein the decontaminant destroys anthrax.

13. The decontaminator of claim 1 further comprising a decontaminant distribution device.

14. A method for decontaminating objects comprising:
storing a decontaminant in a decontaminant container;
regulating flow of the decontaminant from the decontaminant container to a vapor box;
heating the decontaminant in the vapor box to vaporize the decontaminant; and
releasing the vaporized decontaminant into a chamber.

15. The method of claim 14 further comprising regulating flow of the decontaminant into the vapor box via a solenoid valve.

16. The method of claim 14 further comprising disbursing the vaporized decontaminant into the chamber via a vapor propelling device.

17. The method of claim 16 wherein the vapor propelling device is a thermostatic fan.

18. The method of claim 14 further comprising regulating flow of the decontaminant into the vapor box via a valve controlled by a timing relay.

19. The method of claim 16 further comprising controlling the vapor propelling device with a timing relay.

20. The method of claim 14 further comprising closing the chamber with an electromagnetic latch.

21. The method of claim 14 further comprising providing power to at least a portion of the decontaminator via a solar collector.

22. The method of claim 14 wherein the decontaminant is selected from the group consisting of peracetic acid, peroxides, chlorine dioxide, and formaldehydes.

23. The method of claim 22 wherein the decontaminant is hydrogen peroxide.

24. The method of claim 14 wherein the decontaminant destroys anthrax.

25. A mail box decontaminator comprising:
a decontaminant container;
a valve connected to the decontaminant container to regulate flow of decontaminant from the decontaminant container;
a vapor box connected to the valve;
a heater to heat the decontaminant in the vapor box to form a vapor; and
a vapor propelling device to disburse the vapor into the mailbox.

26. The mail box decontaminator of claim 25 further comprising a vapor distribution device.
